(19) United States
(12) Patent Application Publication
(10) Pub. No.: US 2021/0192265 A1
(43) Pub. Date: Jun. 24, 2021

(54) METHOD AND DEVICES FOR ASSESSING THE SUITABILITY OF A SAMPLE TUBE FOR USE IN A LABORATORY AUTOMATION SYSTEM

(71) Applicant: Roche Diagnostics Operations, Inc., Indianapolis, IN (US)

(72) Inventor: Timo Fleischmann, Aichwald (DE)

(73) Assignee: Roche Diagnostics Operations, Inc., Indianapolis, IN (US)

(21) Appl. No.: 17/119,071

(22) Filed: Dec. 11, 2020

(30) Foreign Application Priority Data
Dec. 18, 2019 (EP) 19217673.3

(51) Int. Cl. G06K 9/62; G06K 9/46; G06K 9/52; G06K 9/60; G16H 10/40; G06N 20/00; G01N 35/00

(52) U.S. Cl. CPC G06K 9/627; G06K 9/4652; G06K 9/52; G06K 2209/057; G16H 10/40; G06N 20/00; G01N 35/00663; G06K 9/60

(57) ABSTRACT

A method of assessing the suitability of at least one sample tube for use in a laboratory automation system is disclosed. The method comprises capturing at least one image of at least a part of the sample tube by using a camera of a mobile device, obtaining at least one first item of identification information of the sample tube by analyzing the image, retrieving at least one second item of identification information of the sample tube, and evaluating the first and second items of identification information and assigning the sample tube to at least one suitability category. A mobile device and an assessment system for assessing the suitability of at least one sample tube for use in a laboratory automation system are also disclosed.

### CROSS-REFERENCE TO RELATED APPLICATIONS

This application claims priority to EP 19217673.3, filed Dec. 18, 2019, which is hereby incorporated by reference.

### BACKGROUND

The present disclosure generally relates to a method of assessing the suitability of at least one sample tube for use in a laboratory automation system by using a camera of a mobile device. The present disclosure further relates to an assessment system for assessing the suitability of at least one sample tube for use in a laboratory automation system. Further, the present disclosure refers to a mobile device having at least one camera, to a computer program and a computer-readable storage medium for performing the method. The methods, devices, computer programs and computer-readable storage media specifically may be used in the field of medical or chemical laboratories, for example in order to assess the suitability of a sample tube for use in an analytical or in a pre-analytical laboratory system. Other fields of application of the present invention, however, are feasible.

In the field of medical or chemical laboratories, a wide variety of samples, such as blood, blood serum or plasma, urine or chemical substances, are handled in many different sample tube types. In many cases, the suitability of sample tubes for use in a specific laboratory automation system has to be assessed before use. In some cases, a laboratory automation system autonomously checks the suitability of a specific sample tube for use in the system.

Thus, devices and methods known to the skilled person, which make use of laboratory automation systems comprising, as an example, a camera system to optically identify the sample tubes used in the system are known. However, the advantages achieved by the known methods and devices, specifically in the field of automated optical inspection of the sample tubes, several technical challenges remain. Specifically, the possibility of assessing the suitability of a sample tube for use in a laboratory automation system independent and apart from the system and in advance of use of the sample tube would be desirable. A manual qualification process of unknown sample tubes by qualified technicians in many cases is ineffective as well as time- and cost-consuming.

Therefore, there is a need for a method and devices, which allow for assessing the suitability of a sample tube for use in a laboratory automation system in advance, in a simple and cost-effective manner.

### SUMMARY

According to the present disclosure, a method of assessing the suitability of at least one sample tube for use in a laboratory automation system is presented. The method can comprise capturing at least one image of at least a part of the sample tube by using a camera of a mobile device, obtaining at least one first item of identification information of the sample tube by analyzing the image, retrieving at least one second item of identification information of the sample tube, and evaluating the first and second items of identification information and assigning the sample tube to at least one suitability category.

Accordingly, it is a feature of the embodiments of the present disclosure to provide a method and devices, which allow for assessing the suitability of a sample tube for use in a laboratory automation system in advance, in a simple and cost-effective manner. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates an assessment system, of a mobile device and of a laboratory automation system according to an embodiment of the present disclosure.

FIG. 2 and FIG. 3 illustrate flow charts of different embodiments of a method of assessing the suitability of a sample tube for use in a laboratory automation system according to the present disclosure.

FIG. 4 illustrates a calibration element and of a sample tube according to an embodiment of the present disclosure.

FIGS. 5a-5c illustrate different register cards of a graphical user interface for retrieving at least one second item of identification information according to an embodiment of the present disclosure.

FIG. 6 illustrates a graphical user interface displaying the result of the method according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

As used in the following, the terms “have”, “comprise” or “include” or any arbitrary grammatical variations thereof can be used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms can be used in conjunction with optional features, without restricting alternative possibilities. Thus, features introduced by these terms can be optional features and may not be intended to restrict the scope of the claims in any way. The present disclosure may, as the skilled person will recognize, be performed by using alternative features. Similarly, features introduced by “in an embodiment of the present disclosure” or similar expressions can be intended to be optional features, without any restriction regarding alternative embodiments of the present disclosure, without any restrictions regarding the scope of the present disclosure and without any restriction regarding the possibility of combining the features introduced in such way with other optional or non-optional features of the present disclosure.

A method for assessing the suitability of at least one sample tube for use in a laboratory automation system is disclosed. The method can comprise the following steps, which specifically may be performed in the given order. It can be noted, however, that a different order may also be possible. Further, it can also be possible to perform one or more of the method steps once or repeatedly. Further, it can be possible to perform two or more of the method steps simultaneously or in a timely overlapping fashion. The method may comprise further method steps, which are not listed.

The method can comprise i) capturing at least one image of at least a part of the sample tube by using a camera of a mobile device, ii) obtaining at least one first item of identification information of the sample tube by analyzing the image, iii) retrieving at least one second item of identification information of the sample tube, specifically by the mobile device, and iv) evaluating the first and second items of identification information and assigning the sample tube to at least one suitability category.

The term “assessing the suitability for use” as used herein can be a broad term and can be given its ordinary and customary meaning to a person of ordinary skill in the art and may not be limited to a special or customized meaning. The term specifically may refer, without limitation, to the process of providing at least one item of information on whether or not at least one element or process is suited for being used in conjunction with another element and/or another process, for achieving a specific result. The item of information may be or may comprise a digital information, such as “suited” or “not suited”. Additionally or alternatively, the item of information may be or may comprise a degree of suitability, indicating the extent to which the element or process is suited. Thus, in the context of assessing the suitability of at least one sample tube for use in a laboratory automation system, the assessment specifically may refer, without limitation, to a qualitative and/or quantitative assessment of the suitability of at least one sample tube in an arbitrary and/or a specific laboratory automation system (LAS). The LAS may be configured to handle a sample tube with certain specifications. For example, these specifications may refer to one or more of: the sample tube's geometry, the sample tube's ground shape, the cap type, specifically the cap geometry, the cap color, the cap sealing type, the cap material, and/or the sample tube's material. The result of the assessment may comprise information about the at least one suitability category of the sample tube regarding the specific LAS. For example, the result may comprise information whether or not the sample tube can be compatible with the specific LAS. As another example, the result of the assessment may also comprise information whether the sample tube is of a known type.

The term “suitability category” as used herein can be a broad term and can be given its ordinary and customary meaning to a person of ordinary skill in the art and may not be limited to a special or customized meaning. The term specifically may refer, without limitation, to a classification with regard to the usability of a sample tube in a LAS. The usability of a sample tube may thereby depend on specific requirements of the LAS the sample tube is used for. Thus, the suitability category specifically may reflect and/or comprise the at least one information generated in the assessment. As an example, the suitability category may reflect whether the sample tube is suited for use or is unsuited for use with the LAS or, otherwise, whether the suitability may not be decided and whether additional information on the suitability may have to be retrieved. At least two suitability categories may be defined or pre-defined.

The term “sample tube” as used herein can be a broad term and can be given its ordinary and customary meaning to a person of ordinary skill in the art and may not be limited to a special or customized meaning. The term specifically may refer, without limitation, to a container which can be configured to one or more of contain, store or transport a sample to be analyzed in the LAS. Further, the shape of the container can be such that a tube can be formed, such as a cylindrical tube, e.g., a cylindrical tube having a circular and/or polygonal cross-section. Other types or forms of the container are also possible. The sample tube may comprise a tube bottom, a tube body and a cap, including a sealing of the cap. The tube bottom may be configured to confine the sample tube at the sample tube's lower end. The tube body may be configured to form the shape of the sample tube. The cap may be configured to reversibly close the sample tube at the sample tube's upper end by using a mechanism of a specific sealing type. For example, the sample tube's cap sealing may comprise one or more of a screw type, a rubber type, a hemogard type or a push type. The upper and lower end of the sample tube may be defined by the way of usage of the sample tube.

Further, the term “laboratory automation system” (LAS) as used herein can be a broad term and can be given its ordinary and customary meaning to a person of ordinary skill in the art and may not be limited to a special or customized meaning. The term specifically may refer, without limitation, to a system, which can be configured to handle sample tubes automatically, specifically a system, which can be configured to process sample tubes and their enclosed samples autonomously and/or fully or partially automatically. The laboratory in which the LAS is used may be for example a clinical laboratory, a forensic laboratory or a blood bank. The LAS, as an example, may comprise at least one actuator configured for handling the at least one sample tube, such as configured for handling a plurality of sample tubes, e.g., sequentially or in parallel. The actuator, as an example, may be configured for automatically moving the at least one sample tube, such as through a plurality of handling stations within the system. As an example, the actuator may be or may comprise one or more of a robot arm, a conveying system or a carrousel. Other actuators are known and may be used. The LAS may further comprise one or more handling stations for handling and/or processing one or more samples contained in the at least one sample tube. As an example, one or more stations for adding components to the sample, one or more stations for heating the sample or one or more stations for sample separation such as by centrifuging may be provided. The LAS specifically may be a pre-analytical system, specifically for preparing one or more samples for subsequent sample analysis.

The term “sample” as used herein can be a broad term and can be given its ordinary and customary meaning to a person of ordinary skill in the art and may not be limited to a special or customized meaning. The term specifically may refer, without limitation, to an aliquot of a substance such as a chemical or biological compound. Specifically, the sample may be or may comprise at least one biological specimen, such as one or more of: blood, blood serum, blood plasma, urine and/or saliva. Additionally or alternatively, the sample may be or may comprise a chemical substance or compound and/or a reagent.

The method, as outlined above, can comprise, in step i), capturing at least one image of at least a part of the sample tube by using the camera of the mobile device. The term “mobile device” as used herein can be a broad term and can be given its ordinary and customary meaning to a person of ordinary skill in the art and may not be limited to a special or customized meaning. The term specifically may refer, without limitation, to a mobile electronics device, more specifically to a mobile communication device such as one or more of a mobile phone, a cell phone, a smartphone, a tablet computer, and/or a notebook. Additionally or alternatively, as will be outlined in further detail below, the mobile device may also refer to a tablet computer or another type of portable computer having at least one camera. The mobile device specifically may be portable, such as by having dimensions of less than 5.000 cm³, and/or by having a weight of less than 3 kg.

The term “camera” as used herein can be a broad term and can be given its ordinary and customary meaning to a person of ordinary skill in the art and may not be limited to a special or customized meaning. The term specifically may refer, without limitation, to a device having at least one imaging element configured for recording or capturing spatially resolved one-dimensional, two-dimensional or even three-dimensional optical data or information. As an example, the camera may comprise at least one camera chip, such as at least one CCD chip and/or at least one CMOS chip configured for recording images.

The term “image” as used herein can be a broad term and can be given its ordinary and customary meaning to a person of ordinary skill in the art and may not be limited to a special or customized meaning. The term specifically may refer, without limitation, to data recorded by using a camera, such as a plurality of electronic readings from the imaging device, such as the pixels of the camera chip. Thus, the image may be or may comprise at least one array of information values, such as an array of grey scale values and/or color information values. The image may be a single color image or a multi-color or colored image.

The camera, besides the at least one camera chip or imaging chip, may comprise further elements, such as one or more optical elements, e.g., one or more lenses. As an example, the camera may be a fix-focus camera, having at least one lens, which can be fixedly adjusted with respect to the camera. Alternatively, however, the camera may also comprise one or more variable lenses, which may be adjusted, automatically or manually. The present disclosure specifically may be applicable to cameras as usually used in mobile applications such as notebook computers, tablets or, specifically, cell phones such as smartphones. Thus, specifically, the camera may be part of a mobile device, which, besides the at least one camera, can comprise one or more data processing devices such as one or more data processors. Other cameras, however, can be feasible.

The camera specifically may be a color camera. Thus, such as for each pixel, color information may be provided or generated, such as color values for three colors R, G, B. A larger number of color values may also be feasible, such as four-color values for each pixel. Color cameras are generally known to the skilled person. Thus, as an example, the camera chip may consist of a plurality of three or more different color sensors each, such as color recording pixels like one pixel for red (R), one pixel for green (G) and one pixel for blue (B). For each of the pixels, such as for R, G, B, values may be recorded by the pixels, such as digital values in the range of 0 to 255, depending on the intensity of the respective color. Instead of using color triples such as R, G, B, as an example, quadruples may be used. The color sensitivities of the pixels may be generated by color filters or by appropriate intrinsic sensitivities of the sensor elements used in the camera pixels. These techniques are generally known to the skilled person.

The term “capturing at least one image” as used herein can be a broad term and can be given its ordinary and customary meaning to a person of ordinary skill in the art and may not be limited to a special or customized meaning. The term specifically may refer, without limitation, to one or more of imaging, image recording, image acquisition, and/or image capturing. The term “capturing at least one image” may comprise capturing a single image and/or a plurality of images such as a sequence of images. For example, the capturing of the image may comprise recording continuously a sequence of images such as a video or a movie. The capturing of the at least one image may be initiated by the user action or may automatically be initiated, e.g., once the presence of the at least one object within a field of view and/or within a predetermined sector of the field of view of the camera can automatically be detected. These automatic image acquisition techniques are known e.g., in the field of automatic barcode readers, such as from automatic barcode reading apps. The capturing of the images may take place, as an example, by acquiring a stream or “live stream” of images with the camera, wherein one or more of the images, automatically or by user interaction such as pushing a button, can be stored and used as the at least one first image or the at least one second image, respectively. The image acquisition may be supported by a processor of the mobile device, and the storing of the images may take place in a data storage device of the mobile device.

The method, as outlined above, comprises, in step ii), obtaining at least one first item of identification information of the sample tube by analyzing the image. The term “item of identification information” as used herein can be a broad term and can be given its ordinary and customary meaning to a person of ordinary skill in the art and may not be limited to a special or customized meaning and may not be limited to a special or customized meaning. The term specifically may refer, without limitation, to an arbitrary characteristic, which can be used to identify a sample tube. For example, an item of identification information may describe a geometry, a color, a material and/or a functional feature of the sample tube. The terms “first” and “second” are used for the purpose of nomenclature, only, without ranking or numbering these items of identification information and without giving any preferences. Further, this numbering does not refer to a specific chronological order of the information or to a specific number of information. Specifically, one or more, more specifically two or more than two items of identification may be obtained for both, the first and second item of identification information. The items of identification information may be obtained via different ways. One possible way of obtaining an item of identification information can be by image analysis. Another possible way of obtaining an item of identification information can be by retrieving the information via an interface of the mobile device, as will be outlined in further detail below.

The term “analyzing the image,” also referred to as “image analysis,” as used herein can be a broad term and can be given its ordinary and customary meaning to a person of ordinary skill in the art and may not be limited to a special or customized meaning. The term specifically may refer, without limitation, to an arbitrary process for deriving information from the image. Specifically, in context of step ii), the information obtained by analyzing the image contain at least one first item of identification information. The process of image analysis may be implemented by using a computer vision process. Image analysis techniques are generally known to the skilled person. Thus, as an example, pattern recognition techniques may be used which are generally known, e.g., for detecting the geometry of the sample tube or the like. Other image analysis steps or processes may be used in addition or alternatively. The result of the analyzing of the image may be or may comprise a numerical result and/or another type of information which may directly be used as the first item of identification information and/or which may be used for generating, by further processing, the at least one first item of identification information.

The term “retrieving”, as used herein and as specifically used in the context of step iii), can be a broad term and can be given its ordinary and customary meaning to a person of ordinary skill in the art and may not be limited to a special or customized meaning. The term specifically may refer, without limitation, to the process of actively or passively obtaining one or more of an object and an item of information, specifically data. The retrieving may be initiated by the retrieving entity or by an external entity. The object or item of information to be retrieved may be obtained, as an example, via one or more interfaces, such as a data interface and/or a user interface. As an example, a user may provide the at least one second item of identification information of the sample tube by manually inserting the information, such as in a process of guided data entry, e.g., via one or more queries initiated automatically by the mobile device, e.g., by an App running on the mobile device.

The method, as further outlined above, in step iv), comprises evaluating the first and second item of identification information and assigning the sample tube to at least one suitability category. The term “evaluating” as used herein can be a broad term and can be given its ordinary and customary meaning to a person of ordinary skill in the art and may not be limited to a special or customized meaning. The term specifically may refer, without limitation, to an operation of processing one or more input information/data and thereby obtaining an evaluation result or evaluation quantity, such as one or more of an information, a variable or a parameter. For example, in context of step iv), the first and second item of identification information may be evaluated in order to generate at least one item of information allowing for assigning the sample tube to the at least one suitability category. Examples will be given in further detail below.

The second item of identification information specifically may comprise information other than information obtained by image analysis. Thus, as an example, the second item of information may be or may comprise information, which is not derived or derivable from the image analysis such as one or more items of information selected from the group comprising of information provided by the user, information provided by a database, and/or information provided by another device communicating with the mobile device. As an example, the second item of identification information may be at least partially retrieved via at least one interface of the mobile device. The interface may comprise at least one of a data interface and a user interface, specifically a graphical user interface. The mobile device may prompt the user to input the second item of identification information or at least a part thereof. Thus, the second item of identification information may be at least partially retrieved by a user input, specifically by a manual user input, such as via a user interface of the mobile device. Further, the second item of identification information may also be at least partially retrieved via a software application running on the mobile device, the software application requesting a user of the mobile device to input the second item of identification information, specifically via an input menu, more specifically via having the user select from a plurality of predetermined selectable items. Other ways of retrieving the at least one second item of identification information may be feasible alternatively or additionally.

The term “interface of the mobile device” as used herein can be a broad term and can be given its ordinary and customary meaning to a person of ordinary skill in the art and may not be limited to a special or customized meaning. The term specifically may refer, without limitation, to a feature of the mobile device, which can be configured to interact with its environment, such as for the purpose of unidirectionally or bidirectionally exchanging information, such as for exchange of one or more of data or commands. For example, the interface of the mobile device may be configured to share information with a user and to receive information by the user. The interface of the mobile device may be a feature to interact visually with a user, such as a display, or a feature to interact acoustically with the user. The interface, as an example, may comprise one or more of: a user interface, such as a graphical user interface, and/or a data interface, such as a wireless and/or a wirebound data interface.

The term “software application” as used herein can be a broad term and can be given its ordinary and customary meaning to a person of ordinary skill in the art and may not be limited to a special or customized meaning. The term specifically may refer, without limitation, to a computer program running on a computing unit in order to perform a specific operation. The software application may provide a specific functionality to the computing unit. The computing unit may comprise a computer, a mobile device, a server system.

The term “input menu/user input” as used herein can be a broad term and can be given its ordinary and customary meaning to a person of ordinary skill in the art and may not be limited to a special or customized meaning. The term specifically may refer, without limitation, to a functionality of the software application to record information provided by the user. The input menu may be part of a graphical user interface for retrieving information from the user. Further, the term “user” as used herein can be a broad term and can be given its ordinary and customary meaning to a person of ordinary skill in the art and may not be limited to a special or customized meaning. The term specifically may refer, without limitation, to a person who operates the mobile device, specifically the software application running on the mobile device. The user, as an example, may be a service technician qualified to handle sample tubes and/or a LAS or may be an unexperienced user.

As outlined above, step iv) can comprise evaluating the first and second item identification information and assigning the sample tube to at least one suitability category. This step may comprise combining the first and second item of identification information to obtain at least one combined item of identification information of the sample tube, wherein in step iv), the combined item of identification information can be evaluated. For example, the combined item of identification information may comprise a serial number or an article number of the sample tube. Based on this combined item of identification information, a suitability category may be assigned to the sample tube. Additionally or alternatively, the at least one combined item of identification information may be or may comprise a vector and/or an array of items of information, such as an array comprising both the at least one first item of identification information and the at least one second item of identification information. The combined item of identification information may then be used for further processing, such as for identifying the sample tube and/or for determining the suitability category.

The method may further comprise v) providing the suitability category via at least one interface, specifically via at least one interface of the mobile device, more specifically via at least one display of the mobile device.

Thus, as an example, the software application running on the mobile device may, besides optionally retrieving the at least one second item of identification information, be configured for providing the suitability category via the at least one interface of the mobile device, such as via the at least one graphical user interface, specifically for displaying the suitability on at least one display of the mobile device. It can be noted that other options are also feasible. Thus, as an example, the suitability category may also, additionally or alternatively, be brought to user's attention by other means, such as by audio means and/or haptic means, and/or may be stored in at least one data storage device and/or may be transmitted to at least one other device, such as via the at least one interface, such as to the LAS. The method may also comprise outputting a warning, such as one or more of a visual warning, an audio warning or a haptic warning, such as in case the suitability category may indicate that the sample tube is unsuited for use with the LAS.

As outlined above, the method may comprise capturing and/or determining the sample tube's geometry, such as in step ii). For this purpose, as an example, step i) may comprise using at least one calibration element, wherein the image may comprise at least a part of the calibration element. Thus, as an example, the capturing of the at least one image may take place such that the image shows at least a part of the sample tube and, additionally, at least a part of the calibration element. The calibration element may comprise at least one geometrical reference marker. Further, the image of the at least one part of the calibration element may be used to obtain at least one item of geometrical information of at least a part of the sample tube. The item of geometrical identification information may comprise at least one of: a height of the sample tube, specifically one or more of a height of the sample tube including a cap, a height of a tube body of the sample tube without a cap and a height of the sample tube's cap, more specifically one or more of a height of the sample tube's cap without a nose, a height of the sample tube's cap with nose and a height of the nose of a sample tube's cap; a width of the sample tube, specifically one or more of a width of a sample tube body of the sample tube and a width of a cap of the sample tube; an inner diameter of the sample tube, specifically one or more of an inner diameter of a sample tube body of the sample tube, more specifically one or more of an inner diameter at a predefined position above the sample tube's bottom and an inner diameter at a predefined position below the sample tube's upper edge, and an inner diameter of a cap of the sample tube; an outer diameter of the sample tube, specifically one or more of an outer diameter of a sample tube body of the sample tube, more specifically one or more of an outer diameter at a predefined position above the sample tube's bottom and an outer diameter at a predefined position below the sample tube's upper edge, and an outer diameter of a cap of the sample tube; a shape of at least a part of the sample tube, specifically one or more of a shape of a tube bottom of the sample tube and a shape of a cap of the sample tube; a geometry of a tube bottom of the sample tube, specifically one or more of a radius of a round bottom of the sample tube and a length of a flat bottom of the sample tube; a geometry of a thread of the sample tube, specifically one or more of an outer diameter of a thread of the sample tube, a length of a thread of the sample tube and a number of threads of a thread of the sample tube; an angle of the sample tube's body, specifically one or more of a left angle of the sample tube's body and a right angle of the sample tube's body.

The method of assessing the suitability of the sample tube may also take into account a color information of the sample tube. Therefore, the calibration element may comprise at least one color reference. The image of the at least one part of the calibration element may be used to obtain at least one item of color identification information of at least a part of the sample tube. For example, the item of color identification information may comprise at least one item of color information on a cap of the sample tube. Both items of identification information, the geometrical and color information of the sample tube may be determined by the image analysis in step ii). Therefore, the obtained first item of identification information may comprise at least one of an item of geometrical identification information and/or an item of color identification information.

The term “calibration element” as used herein can be a broad term and can be given its ordinary and customary meaning to a person of ordinary skill in the art and may not be limited to a special or customized meaning. The term specifically may refer, without limitation, to an object of known geometrical and/or color properties, which is used to calibrate one or more of geometrical, dimensional or color information derived from the image. The calibration element, as an example, may be or may comprise at least one substrate, such as a cardboard substrate or a plastic substrate. The substrate may provide calibration information, such as by having one or more of predetermined and known dimensions, color or the like. Additionally or alternatively, the one or more geometrical patterns and/or color patterns may be applied to the substrate, such as printed onto at least one surface of the substrate. As example, the calibration element may comprise at least one printed calibration pattern having known dimensions and/or having known color properties, such as having known R, G or B color coordinated. The calibration element may be used such that the sample tube can be placed onto the calibration element when performing step i), such that both the sample tube and the calibration element can be, at least partially, visible in the image. Additionally or alternatively, the at least one calibration element may be placed next to the sample tube, such that both the sample tube and the calibration element can be at least partially visible in the image. The calibration of the image may, as an example, be performed by comparing the pixel data of the calibration element and the pixel data of the sample tube. The calibration element may also comprise a colored element to calibrate a color scale of the image. For this purpose, the RGB color intensities measured by a color camera may be used. Thus, generally, in step ii), the analysis of the image may take into account at least one item of reference information derived from the image of the calibration element, such as at least one item of geometrical reference information and at least one item of color reference information.

Further, as outlined above, the method can comprise retrieving the at least one second item of identification information in step iii). The second item of identification information may comprise at least one of: an item of material identification information, an item of functional identification information, and/or an item of geometrical identification information. The item of material identification information specifically may comprise at least one of an information on a material of a tube body of the sample tube and/or information on a material of a cap of the sample tube. The item of functional identification information specifically may comprise information on a cap sealing type of a cap of the sample tube. The item of geometrical identification information specifically may comprise information on a tube geometry of at least a part of the sample tube, specifically on one or more of a geometry of a tube bottom of the sample tube and a cap of the sample tube.

As outlined above, the method can comprise in step iv), evaluating the first and second item of identification information. Step iv) specifically may be performed by using at least one algorithm, specifically a machine learning algorithm, more specifically a machine learning classifier, for example a computer vision algorithm. Further in step iv), assigning of the sample tube to at least one suitability category may be performed by using at least one sample tube database. The suitability category may therefore indicate whether the sample tube is of a type already listed in the sample tube database. Thus, the suitability category may comprise at least one category from the group consisting of: “known and compatible”, “unknown, but compatible” and “not compatible”. The suitability category may indicate whether the sample tube is suitable for use with the laboratory automation system. In case the sample tube may be of a type not yet listed in the sample tube database, the sample tube database may be updated. Specifically, the sample tube database may be updated by using at least one result of step iv).

The term “sample tube database” as used herein can be a broad term and can be given its ordinary and customary meaning to a person of ordinary skill in the art and may not be limited to a special or customized meaning. The term specifically may refer, without limitation, to an electronic data management system, which can be configured to handle a specific data structure, wherein the data can refer to sample tubes. The specific data structure may be determined by a database model. The sample tube database may be configured as a qualified sample tube list. For example, the sample tube database may be or may comprise a register, which relates one or more items of identification information to a specific sample tube. The sample tube database may, thus, comprise data on a plurality of sample tube types and additional data, such as data referring to properties of the sample tube types. Further, the sample tube database may comprise suitability information on the sample tube types, such as the suitability category and/or other suitability information relating to the sample tube types listed in the sample tube database.

The term “algorithm” as used herein can be a broad term and can be given its ordinary and customary meaning to a person of ordinary skill in the art and may not be limited to a special or customized meaning. The term specifically may refer, without limitation, to one or more rules for processing input data. For example, the algorithm may be a machine-learning algorithm, which is able to learn the rule of processing by processing data, for example by processing training data.

The mobile device specifically, as outlined above, may comprise at least one processor. The term “processor” as generally used in this description can be a broad term and can be given its ordinary and customary meaning to a person of ordinary skill in the art and may not be limited to a special or customized meaning. The term specifically may refer, without limitation, to an arbitrary logic circuitry configured for performing basic operations of a computer or system, and/or, generally, to a device which can be configured for performing calculations or logic operations. In particular, the processor may be configured for processing basic instructions that drive the computer or system. As an example, the processor may comprise at least one arithmetic logic unit (ALU), at least one floating-point unit (FPU), such as a math coprocessor or a numeric coprocessor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) or the like. The processor specifically may be configured, such as by software programming, for performing one or more evaluation operations.

Further, the processor may be configured to perform step ii) and optionally step iii). The processor may be configured to automatically initiate the capturing of the image in step i), specifically by recognizing if the sample tube can be visible in a field of view of the camera.

Step iv) of the method may be at least partially performed by a backend server remote from the mobile device, specifically by a cloud server. Therefore, the method may comprise transmitting the data obtained in one or more of steps i), ii), and iii) from the mobile device to the backend server. The data obtained in one or both of steps i) and iii) may be recorded via a software application running on the mobile device. The data transmission specifically may take place via one or more wireless and/or wirebound interfaces or networks, such as via the internet.

The term “backend server” as used herein can be a broad term and can be given its ordinary and customary meaning to a person of ordinary skill in the art and may not be limited to a special or customized meaning. The term specifically may refer, without limitation, to a device, which can be capable of performing one or more operations with the data to be processed. Specifically, the server may comprise at least one processor, which may be programmed, by appropriate software, for performing one or more operations with the data to be processed. The at least one backend server may be at least one cloud server.

In a further aspect of the present disclosure, an assessment system for assessing the suitability of at least one sample tube for use in a laboratory automation system is disclosed. The assessment system can comprise at least one mobile device having at least one camera, wherein the mobile device can be configured, specifically by software programming, for capturing at least one image of at least a part of the sample tube by using the camera. The assessment system can be configured, specifically by software programming, for performing the method of assessing the suitability of a sample tube for use in a LAS according to the present disclosure, such as according to any one of the embodiments disclosed above and/or according to any one of the embodiments disclosed in further detail below.

Therefore, the assessment system may further comprise, as outlined above in the context of the method, at least one backend server remote from the mobile device, specifically a cloud server, wherein the backend server may be configured for data exchange with the mobile device, wherein the backend server may be configured, specifically by software programming, for at least partially performing step iv). As outlined above, the assessment system can comprise the mobile phone which may be further configured, specifically by software programming, for performing at least one of steps ii) and iii) of the method of assessing the suitability of a sample tube for use in a LAS.

For further definitions and options of the assessment system, reference may be made to the description of the method in one or more of the embodiments given above and/or according to any one of the embodiments described in further detail below.

In a further aspect of the present disclosure, a mobile device is disclosed, specifically for use in an assessment system according to the present disclosure, such as according to any one of the embodiments disclosed above and/or according to any one of the embodiments disclosed in further detail below. The mobile device can be configured, specifically by software programming, for performing at least steps i), ii) and iii) of the method according to the present disclosure, such as according to any one of the embodiments disclosed above and/or according to any one of the embodiments disclosed in further detail below. The mobile device may be further configured for transmitting data obtained in one or more of steps i), ii), and iii) to a backend server.

For further definitions and options of the mobile device, reference may be made to the description of the method in one or more of the embodiments given above and/or according to any one of the embodiments described in further detail below.

A computer program is also disclosed. The computer program can comprise instructions which, when the program is executed by at least one processor of the assessment system according to the present disclosure, such as according to any one of the embodiments disclosed above and/or according to any one of the embodiments disclosed in further detail below, cause the processor to carry out the method according to the present disclosure, such as according to any one of the embodiments disclosed above and/or according to any one of the embodiments disclosed in further detail below. Similarly, a computer-readable storage medium is disclosed, comprising instructions which, when executed by at least one processor of the assessment system according to the present disclosure, such as according to any one of the embodiments disclosed above and/or according to any one of the embodiments disclosed in further detail below, cause the processor to carry out the method according to the present disclosure, such as according to any one of the embodiments disclosed above and/or according to any one of the embodiments disclosed in further detail below.

As used herein, the term “computer-readable storage medium” specifically may refer to a non-transitory data storage means, such as a hardware storage medium having stored there-on computer-executable instructions. The computer-readable data carrier or storage medium specifically may be or may comprise a storage medium such as a random-access memory (RAM) and/or a read-only memory (ROM).

The computer program may also be embodied as a computer program product. As used herein, a computer program product may refer to the program as a tradable product. The product may generally exist in an arbitrary format, such as in a paper format, or on a computer-readable data carrier and/or on a computer-readable storage medium. Specifically, the computer program product may be distributed over a data network.

The methods and devices according to the present disclosure may provide a large number of advantages over similar methods and devices known in the art. Thus, compared to methods and devices known in the art, the methods and devices described herein may provide the possibility of assessing the suitability of at least one sample tube for use in a laboratory automation system using a mobile device having a camera. Specifically, an immediate evaluation of the sample tube independent and apart from the laboratory automation system may improve flexibility and handling of such an assessment method. Thereby, known sample tubes can be activated immediately for use in a laboratory automation system. If possible, unknown tubes can be qualified immediately and/or potential restrictions can be imposed. Therefore, manual handling and/or manual decanting of sample tubes used in the laboratory automation system may become obsolete.

Further, in case the sample tube has been classified as being unsuitable for the laboratory automation system, it can be thought immediately of an alternative. Thus, the method may enable the consulting staff to evaluate the suitability of a sample tube independent and apart from the laboratory automation system. Advice about sample tubes, which are already qualified and can be used immediately, may be given by non-technician staff. Thus, this method may provide for an easy and comprehensive way of assessing the suitability of sample tubes for use in a laboratory automation system.

As another advantage, which may result from the method using a mobile device, an assessment of the sample tubes by technical staff such as staff from a research and development allowance may become redundant or obsolete. The evaluation may be done automatically by the software application running on the mobile device. Therefore, the method and the assessment system may enable a time- and cost-efficient evaluation of the sample tube.

Further, the method and assessment system of assessing the suitability of the sample tubes may allow a cost-effective and eco-friendly assessment by using a mobile device, since the sample tube to be checked doesn't need unnecessary shipment to an inspection site.

As outlined above, the method may fully, or partially, be computer-implemented. Thus, the method may fully, or partially, be embodied by a software application, such as a software application running on the mobile device, and/or by a software combination comprising at least one software package or software application running on the mobile device and at least one further software package running on the backend server. As indicated above, at least one or more of steps i), ii), and iii) may be computer-implemented or computer-supported by a software application running on the mobile device. Step iv) may be software implemented by one or more of the software application running on the mobile device and/or by a software running on the backend server. The remaining and optional steps may also fully, or partially, be software implemented, as the skilled person will recognize.

A method of assessing the suitability of at least one sample tube for use in a laboratory automation system is presented. The method comprises i) capturing at least one image of at least a part of the sample tube by using a camera of a mobile device, ii) obtaining at least one first item of identification information of the sample tube by analyzing the image, iii) retrieving at least one second item of identification information of the sample tube, specifically by the mobile device, and iv) evaluating the first and second items of identification information and assigning the sample tube to at least one suitability category.

The second item of identification information can comprise information other than information obtained by image analysis. The second item of identification information can be at least partially retrieved via at least one interface of the mobile device. The interface can comprise at least one of a data interface and a user interface such as, for example, a graphical user interface.

In another embodiment, the second item of identification information can be at least partially retrieved by a user input such as, for example, by a manual user input. The mobile device prompts the user to input the second item of identification information.

In another embodiment, the second item of identification information can be at least partially retrieved via a software application running on the mobile device. The software application can request a user of the mobile device to input the second item of identification information such as, for example, via an input menu or via having the user select from a plurality of predetermined selectable items.

Step iv) can comprise combining the first and second items of identification information to obtain at least one combined item of identification information of the sample tube. In step iv), the combined item of identification information can be evaluated.

The method can further comprise v) providing the suitability category via at least one interface such as, for example, via at least one interface of the mobile device or via at least one display of the mobile device.

Step i) can comprise using at least one calibration element. The image can comprise at least a part of the calibration element. The calibration element can comprise at least one geometrical reference marker. The image of the at least one part of the calibration element can be used to obtain at least one item of geometrical information of at least a part of the sample tube. The calibration element can comprise at least one color reference. The image of the at least one part of the calibration element can be used to obtain at least one item of color information of at least a part of the sample tube.

Step iv) can be performed by using an algorithm such as, for example, a machine learning algorithm or a machine learning classifier.

The assigning of the sample tube to at least one suitability category in step iv) can be performed by using at least one sample tube database. The suitability category indicates whether the sample tube is of a type already listed in the sample tube database. The suitability category can comprise at least one category from the group consisting of: “known and compatible”, “unknown, but compatible” and “not compatible”.

The sample tube database can be updated by using at least one result of step iv). The sample tube database can be updated in case the sample tube is of a type not yet listed in the sample tube database.

The suitability category can indicate whether the sample tube is suitable for being used with the laboratory automation system.

The first item of identification information can comprise at least one of: an item of geometrical identification information and/or an item of color identification information.

The item of geometrical identification information can comprise at least one of: a height of the sample tube, specifically one or more of a height of the sample tube including a cap, a height of a tube body of the sample tube without a cap and a height of the sample tube's cap, more specifically one or more of a height of the sample tube's cap without a nose, a height of the sample tube's cap with nose and a height of the nose of a sample tube's cap; a width of the sample tube, specifically one or more of a width of a sample tube body of the sample tube and a width of a cap of the sample tube; an inner diameter of the sample tube, specifically one or more of an inner diameter of a sample tube body of the sample tube, more specifically one or more of an inner diameter at a predefined position above the sample tube's bottom and an inner diameter at a predefined position below the sample tube's upper edge, and an inner diameter of a cap of the sample tube; an outer diameter of the sample tube, specifically one or more of an outer diameter of a sample tube body of the sample tube, more specifically one or more of an outer diameter at a predefined position above the sample tube's bottom and an outer diameter at a predefined position below the sample tube's upper edge, and an outer diameter of a cap of the sample tube; a shape of at least a part of the sample tube, specifically one or more of a shape of a tube bottom of the sample tube and a shape of a cap of the sample tube; a geometry of a tube bottom of the sample tube, specifically one or more of a radius of a round bottom of the sample tube and a length of a flat bottom of the sample tube; a geometry of a thread of the sample tube, specifically one or more of an outer diameter of a thread of the sample tube, a length of a thread of the sample tube and a number of threads of a thread of the sample tube; an angle of the sample tube's body, specifically one or more of a left angle of the sample tube's body and a right angle of the sample tube's body.

The item of color identification information can comprise at least one item of color information on a cap of the sample tube.

The second item of identification information can comprise at least one of an item of material identification information and/or an item of functional identification information and/or an item of geometrical identification information.

The item of material identification information can comprise at least one of information on a material of a tube body of the sample tube and/or information on a material of a cap of the sample tube.

The item of functional identification information can comprise information on a cap sealing type of a cap of the sample tube.

The item of geometrical identification information can comprise information on a tube geometry of at least a part of the sample tube such as, for example, on one or more of a geometry of a tube bottom of the sample tube and a cap of the sample tube.

The mobile device can comprise at least one processor. The processor can be configured to perform step ii) and optionally step iii). The processor can be configured to automatically initiate the capturing of the image in step i) such as, for example, by recognizing if the sample tube is visible in a field of view of the camera.

Step iv), at least partially, can be performed by a backend server remote from the mobile device such as, for example, by a cloud server.

The method can further comprise transmitting data obtained in one or more of steps i), ii), and iii) from the mobile device to the backend server.

Data obtained in one or both of steps i) and step iii) can be recorded via a software application running on the mobile device.

An assessment system for assessing the suitability of at least one sample tube for use in a laboratory automation system is disclosed. The assessment system can comprise at least one mobile device having at least one camera. The mobile device can be configured such as by software programming to capture at least one image of at least a part of the sample tube by using the camera. The assessment system can be configured such as by software programming to perform the above method.

The assessment system can further comprise at least one backend server remote from the mobile device such as, for example, a cloud server. The backend server can be configured to data exchange with the mobile device. The backend server can be configured such as by software programming to, at least partially, perform step iv).

The mobile phone can further be configured such as by software programming to perform at least one of steps ii) and iii).

A mobile device such as, for example, for use in an above assessment system. The mobile device can be configured such as by software programming to perform at least steps i), ii) and iii) of the above method.

The mobile device can further be configured to transmit data obtained in one or more of steps i), ii), and iii) to a backend server.

A computer program comprising instructions, which, when the program is executed by at least one processor of the above assessment system, can cause the processor to carry out the above method.

A computer-readable storage medium comprising instructions which, when executed by at least one processor of the above assessment system, can cause the processor to carry out the above method.

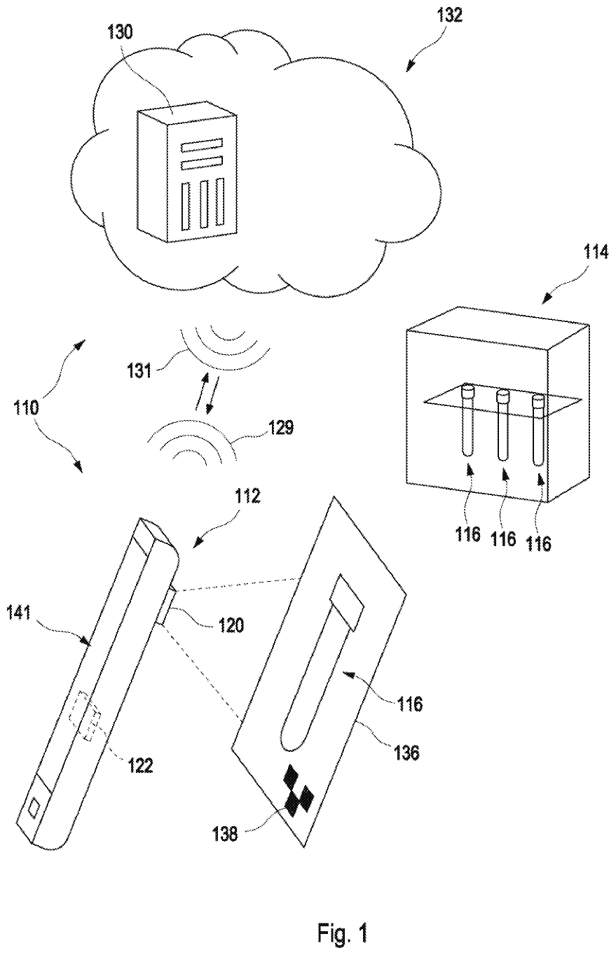

Referring initially to FIG. 1, in FIG. 1, embodiments of an assessment system 110, of a mobile device 112 and of a laboratory automation system 114 according to the present disclosure are shown in a schematic view.

The assessment system 110 can comprise the at least one mobile device 112 and can be configured such as by software programming to perform a method 118 of assessing the suitability of at least one sample tube 116 for use in the laboratory automation system 114. The assessment system 110 can further comprise at least one backend sever device 130, such as at least one cloud server 132, as will be outlined in further detail below. The mobile device 112 can have at least one camera 120 and may comprise at least one processor 122.

The assessment system 110 may further comprise at least one calibration element 136. The calibration element 136 may comprise at least one geometrical reference marker 138 and/or at least one color reference, as will be explained in further detail below.

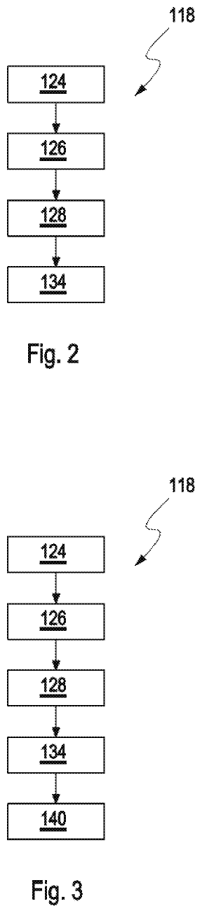

The assessment system 110 can be configured to perform a method of assessing the suitability of the at least one sample tube 116 for use in the laboratory automation system 114. In FIG. 2, a flow chart of an exemplary embodiment of the method is shown, wherein the method is denoted by reference number 118. The method 118 can comprise the following steps, which may specifically be performed in the given order. Still, a different order may also be possible. It may be possible to perform two or more of the method steps fully or partially simultaneously. It may further be possible to perform one, more than one or even all of the method steps once or repeatedly. The method 118 may comprise additional method steps that are not listed. The method steps of the method 118 are the following:
i) (denoted with reference number 124) capturing at least one image of at least a part of the sample tube 116 by using the camera 120 of the mobile device 112;
ii) (denoted with reference number 126) obtaining at least one first item of identification information of the sample tube 116 by analyzing the image;
iii) (denoted with reference number 128) retrieving at least one second item of identification information of the sample tube 116, specifically by the mobile device 112; and
iv) (denoted with reference number 134) evaluating the first and second items of identification information and assigning the sample tube 116 to at least one suitability category.

The image captured in step i) 124 of the method 118 may comprise at least a part of the calibration element 136. In step ii) 126, the first item of identification information may comprise at least one of an item of geometrical identification information and/or an item of color identification information. The calibration element 136 may be used to obtain the item of geometrical identification information and/or the item of color identification information of at least a part of the sample tube 116. For example, the geometrical item of identification information may comprise at least one of: a height of the sample tube 116; a width of the sample tube 116; an inner diameter of the sample tube 116; an outer diameter of the sample tube 116 and/or a shape of the sample tube 116. The item of color identification information may comprise, for example, at least one item of color information on a cap of the sample tube 116. Further, the first item of identification information can be obtained by image analysis. For example, the image analysis may be implemented by a computer vision process. Other image analysis steps or processes may be used in addition or alternatively.

In step iii) 128 of the method 118, a second item of identification information can be retrieved, whereby the second item of identification information may comprise information other than information obtained via image analysis. The second item of identification information may comprise at least one of: an item of material identification information; an item of functional identification information and/or an item of geometrical identification information. The item of material identification information may comprise information on a material of a tube body and/or of a cap of the sample tube 116. The item of functional identification information may comprise information on a cap sealing type of the cap of the sample tube 116. The item of geometrical identification information may comprise information on a geometry of at least a part of the sample tube 116 such as, for example, on one or more of a geometry of a tube bottom and/or of a cap of the sample tube 116.

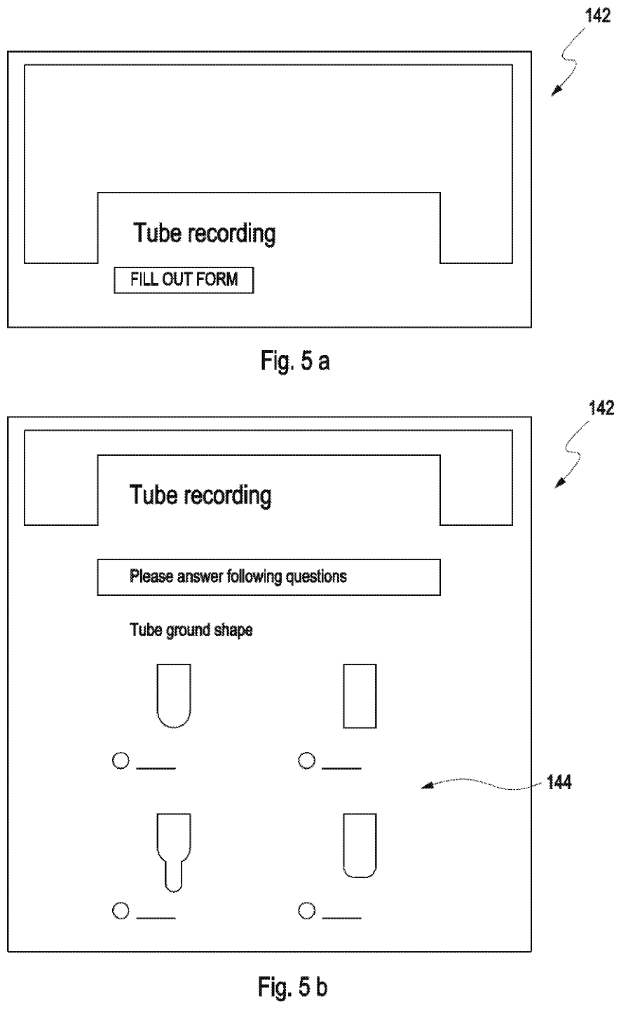

Further, the second item of identification information may be retrieved via an interface of the mobile device 112, wherein the interface may comprise at least one of a data interface and/or a user interface such as, for example, a graphical user interface. The mobile device 112 may prompt a user to input the second item of identification information. For example, the second item of identification information may be retrieved by a user input such as, for example, via a manual user input. An exemplary embodiment of different register cards of the graphical user interface is shown in FIGS. 5a-c and will be described in further detail below.

In step iv) 134, the first and second items of identification information can be evaluated and the sample tube 116 can be assigned to at least one suitability category. Step iv) 134 may comprise combining the first and second items of identification information to obtain at least one combined item of identification information of the sample tube 116, wherein, in step iv) 134, the combined item of identification information may be evaluated. The combined item of identification information may comprise a serial number or an article number of the sample tube 116. Additionally or alternatively, the at least one combined item of identification information may be or may comprise a vector and/or an array of items of information, such as an array comprising both the at least one first item of identification information and the at least one second item of identification information. The combined item of identification information may then be used for further processing, such as for identifying the sample tube 116 and/or for determining the suitability category. Further, step iv) 134 may be performed by using an algorithm such as a machine learning algorithm or a machine learning classifier. The assigning of the sample tube 116 to at least one suitability category may be performed by using at least one sample tube database. The suitability category may indicate whether the sample tube 116 is of a type already listed in the sample tube database and furthermore or whether the sample tube 116 is suitable for use with the laboratory automation system 114.

As outlined above, the processor 122 of the mobile device 112 may be configured to initiate capturing the image in step i) 124 and to perform step ii) 126 and optionally step iii) 128. Further, step iv) 134 may be at least partially performed by the backend server 130 remote from the mobile device 112 such as by the cloud server 132. Therefore, the method 118 may comprise transmitting the data obtained in one or more of steps i) 124, ii) 126 or iii) 128 from the mobile device 112 to the backend server 130.

In FIG. 3, a flow chart of a further embodiment of a method of assessing the suitability of the at least one sample tube 116 for use in the laboratory automation system 114 is shown. The embodiment widely corresponds to the embodiment of FIG. 2. Further, however, as illustrated by FIG. 3, the method 118 may comprise an additional step v) 140, in which the suitability category can be provided via at least one interface such as, for example, via at least one interface of the mobile device 112 such as a user interface, for example, via at least one display of the mobile device 112.

Turning back to FIG. 1, the mobile device 112 can be configured such as by software programming to perform at least steps i) 124, ii) 126 and iii) 128 of the method 118, such as according to any one of FIG. 2 or 3. In step i) 124 of the method 118, the mobile device 112 can be used to capture at least one image of at least a part of the sample tube 116 by using the camera 120 of the mobile device 112. The processor 122 of the mobile device 112 may be configured to automatically initiate the capturing of the image such as, for example, by recognizing if the sample tube 116 is visible in a field of view of the camera 120. As example, a “live” image of a scene visible in the field of view of the camera 120 may be evaluated, e.g., continuously, in order to detect the presence of the sample tube 116. However, another way of triggering the image capture can be also feasible, such as a manual triggering. Further, the processor 122 may be configured to perform step ii) 126 and optionally step iii) 128 of the method 118.

The mobile device 112 may further be configured to transmit the data obtained in one or more of steps i) 124, ii) 126 and iii) 128 from the mobile device 112 to a backend server 130 remote from the mobile device 112. For this purpose, as an example, at least one interface 129 of the mobile device 112 may be used, such as a wireless interface, communicating with at least one interface 131 of the backend server 130. Further, the backend server 130 may be configured such as by software programming for at least partially performing step iv) 134.

Further, step i) 124 of the method 118 may comprise using the at least one calibration element 136, wherein the image may comprise at least a part of the calibration element 136. The calibration element 136 may comprise at least one geometrical reference marker 138 and/or at least one color reference.

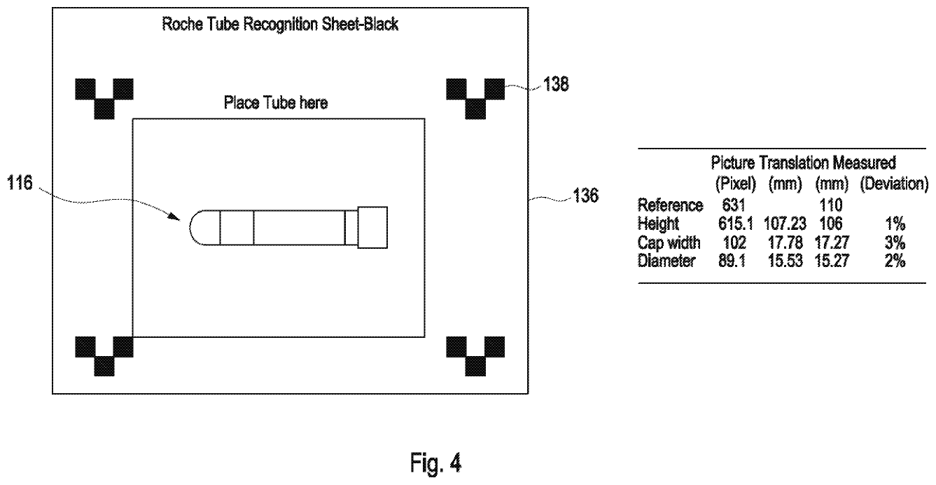

FIG. 4 shows an exemplary embodiment of the calibration element 136 and of the sample tube 116. The calibration element 136 may comprise one or more of a geometrical reference marker 138 and/or a color reference. The geometrical reference marker 138 may have known geometrical properties and may be used to calibrate one or more of geometrical and/or dimensional information derived from the image. The color reference may have known color properties and may be used to calibrate color information derived from the image. The sample tube 116 may be placed onto the calibration element 136 while performing step i) 124 of the method 118. Additionally, or alternatively, the calibration element 136 may also be placed next to the sample tube 116, such that both the sample tube 116 and the calibration element 136 may be at least partially visible in the image. The calibration element 136 may have a high contrast compared to the sample tube. Specifically, the calibration element 136 may have a dark background if the sample tube comprises bright colors, or vice versa.

As outlined above, display 141 may be used as a graphical user interface, e.g., by allowing displaying information and/or by allowing a user to input data via the display 141, such as via a touch screen. Additionally, or alternatively, the mobile device 112 may comprise further user interfaces. The method 118, as outlined above, may be computer-implemented, such as at least partially by an application or App running on the mobile device 112 and, additionally, software running on the cloud server 132.

In FIGS. 5a-c, different register cards of an exemplary embodiment of the graphical user interface are illustrated, as may be generated by the App executed by the processor 122 of the mobile device 112. As outlined above, in step iii) 128 of the method 118, the second item of identification information can at least partially be retrieved via a graphical user interface. For example, the second item of identification information may at least partially be retrieved via a software application running on the mobile device 112, the software application requesting a user of the mobile device 112 to input the second item of identification information such as via an input menu 142 or via having the user select from a plurality of predetermined items 144. The plurality of predetermined items 144 may comprise one or more of: a material of a tube body of the sample tube 116; a material of a cap of the sample tube 116; a cap sealing type of a cap of the sample tube 116; a geometry of a tube bottom of the sample tube 116; and/or a geometry of a cap of the sample tube 116. Thus, in FIG. 5a, the mobile device 112 may start the request to input the second item of identification, by requesting the user to fill out a form as shown in the subsequent images. In FIGS. 5b and 5c, input menus 142 can be shown, requesting the user to select from the plurality of predetermined items 144. FIG. 5b shows the request for selection from a plurality of predetermined tube bottoms, also referred to as tube ground shapes. FIG. 5c shows the request for selection from a plurality of predetermined cap sealing types.

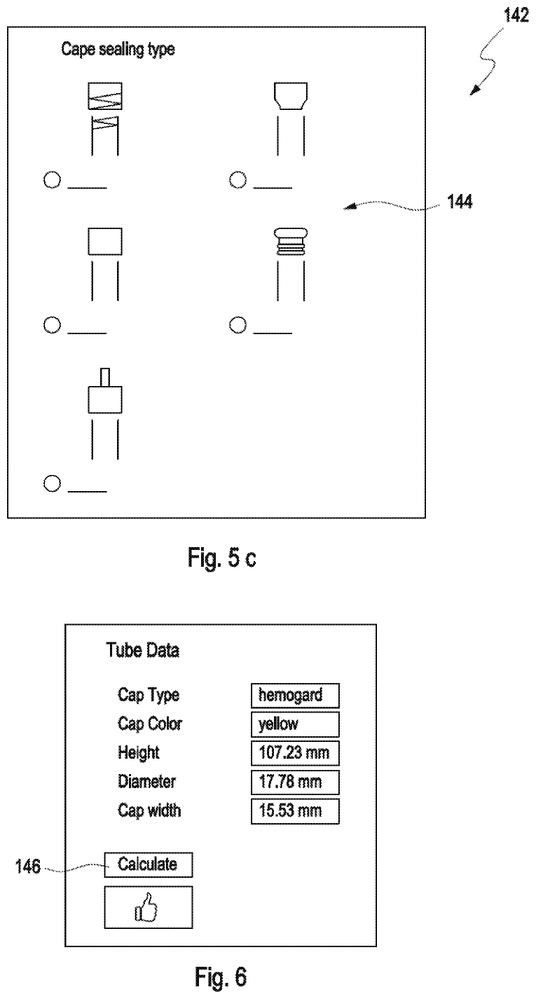

Method 118 may generally comprise showing or displaying at least one result, such as the at least one suitability category, such by using the display 141 of the mobile device 112. Thus, FIG. 6 shows an exemplary embodiment of a graphical user interface displaying the result of the method 118. The result of the method 118 may comprise the suitability category which can be assigned to the sample tube 116 in step iv) 134 of the method 118. The suitability category may comprise at least one category from the group consisting of: “known and compatible”, “unknown, but compatible” and “not compatible”. As outlined above, the suitability category may be provided via at least one interface such as via at least one interface of the mobile device 112 or via at least one display of the mobile device 112. For example, the software application running on the mobile device 112 may further be configured to provide the suitability category via the at least one interface of the mobile device 112, such as via the at least one graphical user interface, for example, for displaying the suitability on at least one display of the mobile device 112. Other options are also feasible. In FIG. 6, as an example, firstly, the tube data of sample tube 116 can be displayed, such as the cap type, the cap color, the height, the diameter, and the cap width. This summary may allow the user to correct data or add missing data. Further, an interactive button 146 may be displayed, allowing a user to actively start the evaluation, based on the tube data listed above. Having started the evaluation, at least one result may be shown, such as the suitability category. Thus, as an example shown in FIG. 6, the “thumbs-up” symbol may indicate that the sample tube 116 is “known and compatible” for use with the laboratory automation system 114. Other symbols and/or text may be used for displaying the result. Further, besides the at least one suitability category, additional information may also be displayed.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A method of assessing the suitability of at least one sample tube for use in a laboratory automation system, the method comprising:
i) capturing at least one image of at least a part of the sample tube by using a camera of a mobile device;
ii) obtaining at least one first item of identification information of the sample tube by analyzing the image;
iii) retrieving at least one second item of identification information of the sample tube; and
iv) evaluating the first and second items of identification information and assigning the sample tube to at least one suitability category.

2. The method according claim 1, wherein the second item of identification information is at least partially retrieved by a user input.

3. The method according to claim 2, wherein the mobile device prompts the user to input the second item of identification information.

4. The method according to claim 1, further comprising,
v) providing the suitability category via at least one display of the mobile device.

5. The method according to claim 4, wherein step i) comprises using at least one calibration element, wherein the image comprises at least a part of the calibration element.

6. The method according to claim 1, wherein step iv) is performed by using a machine learning algorithm.

7. The method according to claim 1, wherein the assigning of the sample tube to at least one suitability category in step iv) is performed by using at least one sample tube database.

8. The method according to claim 1, wherein the first item of identification information comprises at least one of: an item of geometrical identification information and/or an item of color identification information.

9. The method according to claim 1, wherein the second item of identification information comprises at least one of: an item of material identification information, an item of functional identification information, and/or an item of geometrical identification information.

10. The method according to claim 1, wherein step iv) is, at least partially, performed by a backend server remote from the mobile device.

11. An assessment system for assessing the suitability of at least one sample tube for use in a laboratory automation system, the assessment system comprising:
at least one mobile device having at least one camera, wherein the mobile device is configured for capturing at least one image of at least a part of the sample tube by using the camera, wherein the assessment system is configured to perform the method according to claim 1.

12. The assessment system according to claim 11, further comprising,
at least one backend server remote from the mobile device, wherein the backend server is configured for data exchange with the mobile device, wherein the backend server is configured to, at least partially, perform step iv).

13. A mobile device, wherein the mobile device is configured to perform at least steps i), ii) and iii) of the method according to claim 1.

14. The mobile device according to claim 13, wherein the mobile device is further configured to transmit data obtained in one or more of steps i), ii), and iii) to a backend server.

15. A non-transitory computer-readable storage medium comprising instructions which, when executed by at least one processor of the assessment system according to claim 11, cause the processor to carry out the method according to claim 1.
